(19) United States
(12) Plant Patent Application Publication
(10) Pub. No.: US 2006/0041966 P1
(43) Pub. Date: Feb. 23, 2006

(54) White oak tree named AFT-O2

(50) QUERCUS ALBA; WHITE OAK

(76) Inventor: Walter F. Beineke, West LaFayette, IN (US)

Correspondence Address: BARNES & THORNBURG, LLP, P.O. BOX 2786, CHICAGO, IL, 60690-2786, US

(21) Appl. No.: 10/919,902

(22) Filed: Aug. 17, 2004

(51) Int. Cl. A01H 5/00

(52) U.S. Cl. PLT/225

(57) ABSTRACT

A new and distinct cultivar of white oak tree (Quercus alba L.) which is distinctly characterized by extremely rapid growth rate and excellent strength thereby producing good timber qualities. This new variety of white oak trees was discovered by the applicant near Vallonia, Jackson County, Ind. in the front yard of a home on land that had been owned since 1875. This selection has been designated as WO43 in records maintained by the applicant on the performance of this selection, and grafts made from the selection and will be known henceforth as ‘AFTO-2’.

### LATIN NAME OF THE GENUS AND SPECIES

Quercus alba L.

### VARIETY

‘AFTO-2’

### RELATED APPLICATIONS

U.S. Ser. No. 10/919,761

and

U.S. Ser. No. 10/919,574

### BACKGROUND OF THE INVENTION

This new variety of white oak tree (Quercus alba L.) was discovered by the applicant near Vallonia, Jackson County, Ind. in a front yard of a home. It was selected because of its appearance which was consistent with excellent timber quality and progeny tested to confirm genetic reproducibility. This selection has been designated as WO43 in records maintained by the applicant on the performance of this selection, and grafts made from the selection and will be known henceforth as ‘AFTO-2’. Both parents are unknown.

Before the house was constructed in the 1930's, the area was a wooded pasture with many white oak and hickory trees present. The trees were open-grown and the trees developed large, spreading crowns. The U.S. government first sold the land to a Peters family in the 1850's. Therefore, the tree was started over 35 years after the land was settled and used as a farm. The soil is almost pure sand and a part of a line of old sand dunes in the area. The soil is a Princeton sand and very droughty. Therefore, it seems that this tree (the largest in the area) was able to grow extremely fast in spite of the dry environment, indicating a superior genetic trait for rapid growth.

In progeny test plantations at two separate sites consisting of a total of nearly 3,700 trees from 70 seed sources, seedlings from ‘AFTO-2’ have consistently outgrown progeny of the 68 other seed sources, thereby proving that this tree has superior genetic growth traits. Seedlings from ‘AFTO-2’ are extremely fast growing having ranked first (tied with ‘AFTO-3’, which is the subject of a separate patent application) out of the 70 families in the two progeny tests on diverse sites. The percent improvement in growth over the plantation average for the two tests was 18 and 21 percent. That is, at age 10, ‘AFTO-2’ seedlings averaged 27.0 ft. tall at the first planting (plantation average was 22.9 ft) and therefore an improvement of 18% was obtained. At the second planting, ‘AFTO-2’ seedlings averaged 11.5 ft. tall (plantation average was 9.5 ft.) and therefore an improvement of 21 % was obtained.

### SUMMARY OF THE INVENTION

A new and distinct cultivar of white oak tree (Quercus alba L.) is distinctly characterized by extremely rapid growth rate, strong central stem tendency, and excellent straightness, thereby producing excellent timber qualities, the trait of commercial interest. ‘AFTO-2’ was approximately 118 years old when described at a location near Vallonia, Ind. in a cultivated state. Real estate records show that the land was settled and farmed for at least 35 years before the tree was started.

After the original clone was selected, and assigned an identity number of WO43 the aforesaid tree was reproduced by collecting scions from it and grafting these onto common white oak rootstocks at American Forestry Technologies, Inc., West Point, Ind. These asexual reproductions ran true to the originally discovered tree and to each other in all respects.

Color values used were from the Munsell Color Chart for Plant Tissues.

No disease or insect problems were detected.

‘AFTO-2’ is hardy in USDA zones 5, 6, 7, and 8.

### DESCRIPTION OF THE DRAWINGS

FIG. 1 is a photograph showing the timber form of ‘AFTO-2’.

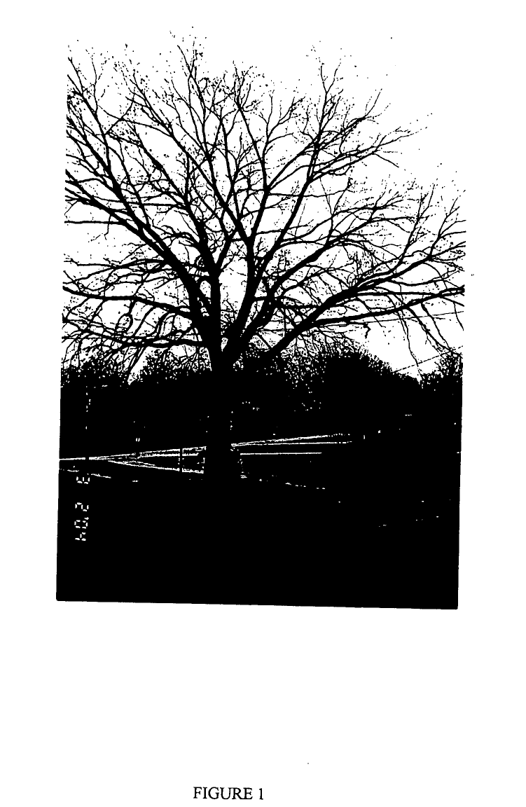

FIG. 2 is a photograph showing the leaves and nuts of ‘AFTO-2’.

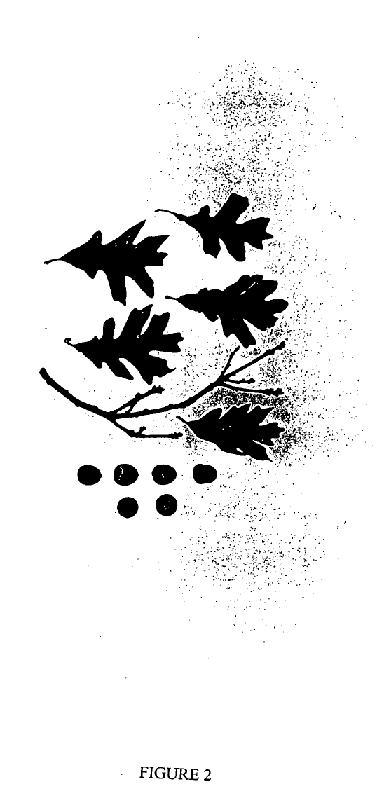

FIG. 3 is a photograph showing the bole of ‘AFTO-2’.

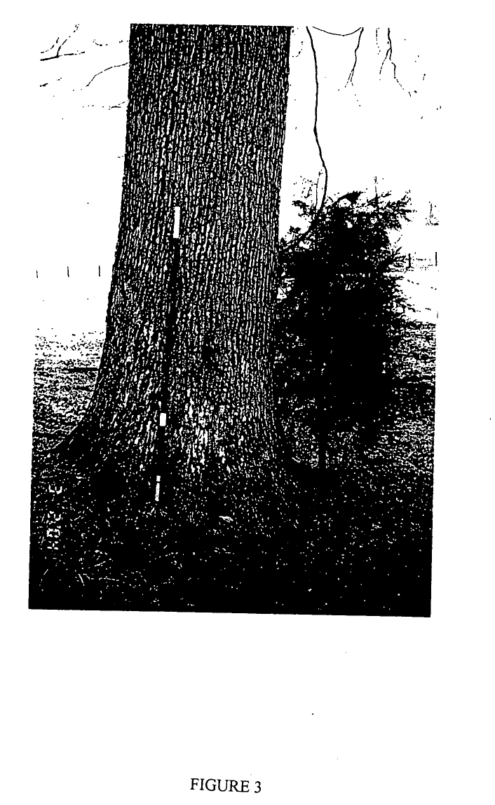

### BOTANICAL DESCRIPTION OF THE PLANTS

The botanical details of this new and distinct variety of white oak tree are as follows. Comparisons for some traits can only be made among ‘AFTO-1’ (U.S. Ser. No. 10/919,761) ‘AFTO-2’ (U.S. Ser. No. 10/919,902) and ‘AFTO-3’ (U.S. Ser. No. 10/919,574) because no comparable measurements are available on other white oak trees except for growth.
Tree:
   Size.—Large, 65 ft. tall; crown diameter of 110 ft.
   Vigor.—Vigorous.
   Growth rate.—Very rapid. Diameter at 4½ feet above the ground at 118 years was 47.0 inches for an average growth rate of 0.40 inches per year. For an old tree this is extremely rapid growth.
   Form.—Excellent timber form. ‘AFTO-2’ rates 1 on the 1 to 5 scale. Stem form was obtained by subjectively rating the straightness of the main stem on a scale of 1 to 5 with 1 representing a perfectly straight stem; 2, slight crook or deviation of the central stem (no crooks); 3, about average straightness; 4, several severe crooks or a single fork; and 5, a very crooked, forked and/or leaning central stem.
Branches and trunk:
   Branch and trunk color.—One year old branches, deep reddish — 7.5R5/4 on the Munsell Color Chart for Plant Tissues; Older branches, dark yellow-green — 7.5GY4/2 on the Munsell Color Chart for Plant Tissues; mature trunk and branches, mottled grays — 7.5YR5/2 and 2.5Y7/2 on the Munsell Color Chart for Plant Tissues.
   Branch and trunk characteristics.—One year old branches, slender, glabrous; older branches, smooth; mature trunk and branches, flaky, loose edges, ridged and furrowed, typical of the species.
   Internode length.—Average; 0.8 inches.
   Branch lenticels.—Are round and average 0.031 inches in diameter. Light yellow-gray — 2.5Y8/2 on the Munsell Color Chart for Plant Tissues.
Leaves:
   Leaves.—Size — Medium; average length including petiole — 5.88″; average width 3.62″. Leaves of ‘AFTO-2’ are larger than ‘AFTO-3’ and smaller than ‘AFTO-1’. ‘AFTO-2’ leaves average over ¾ inch wider and ¾ inch longer than ‘AFTO-3’. ‘AFTO-2’ averages ½ inch narrower and nearly 2 inches shorter than ‘AFTO-1’. The leaf base of ‘AFTO-2’ is very acute compared to either ‘AFTO-1’ or ‘AFTO-3’.
   Thickness.—Thick.
   Texture.—Upper surface, smooth, glabrous; Lower surface, glabrous with white bloom.
   Margins.—Lobed, entire.
   Color.—Upper Surface — dark green (2.5G4/6 by the Munsell Color Chart for Plant Tissues); Lower surface — light green (2.5G6/4 on the Munsell Color Chart for Plant Tissues).
   Average distance between sinuses at leaf center.—0.93″. The sinuses of ‘AFTO-2’ (0.93 inches) are deeper (i.e. closer to the midrib) than ‘AFTO-1’ (1.38 inches), but are slightly shallower than ‘AFTO-3’ (0.78 inches).
   Average number of lobes.—7.0.
   Lobes.—Rounded, typical of species. ‘AFTO-2’ averages 7.0 lobes, slightly more than ‘AFTO-1 (6.8) and fewer than ‘AFTO-3’ (7.8).
   Petioles.—Length — ‘AFTO-2’ has very long petioles (0.78 inches) compared to its leaf length, and the petioles are longer than either ‘AFTO-1’ (0.64 inches) or ‘AFTO-3’ (0.52 inches). Color — reddish (10R4/4 on the Munsell Color Chart for Plant Tissues).
Buds:
   Buds.—Typical of species — rounded, many imbricate scales, glabrous, smaller than average — 0.20″. The buds of ‘AFTO-2’ are about the same length as ‘AFTO-3’ and shorter than ‘AFTO-1’. Color — brownish red (5YR4/4 on the Munsell Color Chart for Plant Tissues).
Acorn:
   Nut shape.—Very large for the species, broad barrel shape; length without cap — 1.0″, diameter 0.92 inches; Cap — width 1.02″, depth, very shallow, 0.24″. Acorns of ‘AFTO-2’ are nearly ¼ inch larger in diameter than ‘AFTO-3 and about the same length as ‘AFTO-3’. Thus the acorns of ‘AFTO-2 are very large and barrel shaped while ‘AFTO-3’ acorns are long and narrow. The acorn cups of ‘AFTO-2’ are very wide — over ¼ inch wider than ‘AFTO-3’. Acorns ripen two weeks earlier than ‘AFTO-3’.
   Flowering habit.—Flowers annually — unusual in the species. Flowers around May 10.
   Fruiting habit.—Produces annual and usually abundant acorn crops. This is very unusual in white oak which usually produces acorns only once every 3 to 5 years. Acorns ripen and fall on about October 8 — much earlier than most white oaks.
   Male flowers.—Staminate aments are pendent. Clustered individual male flowers comprise of 5 lobed calyx which encloses an average of 8.2 stamens (much higher than most white oaks, which normally have 6 stamens). Staminate aments average 2.2 inches long and contain an average of 24.3 flowers or a total of 199.3 stamens per ament. Each stamen contains several thousand pollen grains. Color is a bright yellow-green 2.5 GY 8/12. The male flowers of ‘AFTO-2’ are longer by 1/2 inch and average about 7 more flowers than ‘AFTO-3’.

Female flowers.—Pistillate flowers consist of tiny apetallate spikes in the axils of new leaves. Individual flowers have a 6 lobed calyx. Number of pistillate flowers in each flowering axil vary from 2-4 and average 2, 8. Spikes average 0.12 inches long and are reddish in color — 10R 4/6.

| | LEAVES | | | | | |
|---|---|---|---|---|---|---|
| | | | Distance | | | |
| | | | between | | Petiole | BUDS |
| | Length | Width | sinuses | Number | Length | Length |
| | (inches) | (inches) | (inches) | of lobes | (inches) | (inches) |
| AFTO- | 7.73 | 4.17 | 1.38 | 6.80 | 0.64 | 0.30 |
| 1 | | | | | | |
| AFTO- | 5.88 | 3.62 | 0.93 | 7.00 | 0.78 | 0.20 |
| 2 | | | | | | |
| AFTO- | 5.10 | 2.78 | 0.78 | 7.80 | 0.52 | 0.21 |
| 3 | | | | | | |

| | | | | | | MALE | |
|---|---|---|---|---|---|---|---|
| | ACORNS | | | ACORN CUPS | | FLOWERS | |
| | | | Rip- | | | | |
| | | Dia- | en- | | | | Number |
| | Length | meter | ing | Width | Depth | Length | of |
| | (inches) | (inches) | date | (inches) | (inches) | (inches) | flowers |
| AFTO- | — | — | — | — | — | — | — |
| 1 | | | | | | | |
| AFTO- | 1.00 | 0.92 | Oct- | 1.02 | 0.24 | 2.20 | 24.30 |
| 2 | | | ober | | | | |
| | | | 8 | | | | |
| AFTO- | 1.10 | 0.74 | Oct- | 0.71 | 0.27 | 1.80 | 17.50 |
| 3 | | | ober | | | | |
| | | | 20 | | | | |

### DOCUMENTS CITED

O'Connor, Philip and Beineke, Walter F. (2004) “White Oak seedling performance: is seed source important?” Woodland Steward, vol. 13, no. 1, pp. 10-11, 13 and 15.

I claim:

1. A new and distinct variety of white oak tree named ‘AFTO-2’ substantially as illustrated and described, which has excellent timber quality, extremely rapid growth rate, and fairly strong central stem tendency, and produces annual acorn crops.
